(19) United States
(12) Patent Application Publication
(10) Pub. No.: US 2015/0132382 A1
(43) Pub. Date: May 14, 2015

(54) COMPOSITIONS AND METHODS OF TREATMENT COMPRISING FOSFOMYCIN DISODIUM

(71) Applicant: Forest Laboratories Holdings Ltd., Hamilton (BM)

(72) Inventors: Mahendra G. Dedhiya, Pomona, NY (US); Sisir Bhattacharya, Commack, NY (US)

(21) Appl. No.: 14/537,143

(22) Filed: Nov. 10, 2014

Related U.S. Application Data
(60) Provisional application No. 61/902,354, filed on Nov. 11, 2013.

(51) Int. Cl. A61K 31/665; A61K 45/06

(52) U.S. Cl. CPC A61K 31/665; A61K 45/06
USPC 424/482; 549/217; 514/99; 514/2.9; 424/497

(57) ABSTRACT

The present invention provides compositions comprising fosfomycin or a pharmaceutically acceptable salt, solvate or prodrug thereof (e.g., fosfomycin disodium) alone or in combination with an antibacterial agent. The present invention provides methods of treating bacterial infections, which include administering an effective amount of fosfomycin or a pharmaceutically acceptable salt, solvate or a prodrug thereof (e.g., fosfomycin disodium) alone or in combination with an antibacterial agent.

### CROSS-REFERENCE TO RELATED APPLICATIONS

This application claims priority under 35 U.S.C. §119, based on U.S. Provisional Application Ser. No. 61/902,354 filed on Nov. 11, 2013, which is hereby incorporated by reference in its entirety.

### FIELD OF THE INVENTION

The present invention relates to compositions comprising fosfomycin or a pharmaceutically acceptable salt, solvate or prodrug thereof (e.g., fosfomycin disodium) alone or in combination with an antibacterial agent and methods of treating bacterial infections comprising administering fosfomycin or a pharmaceutically acceptable salt, solvate or prodrug thereof (e.g., fosfomycin disodium) alone or in combination with an antibacterial agent.

### BACKGROUND OF THE INVENTION

Fosfomycin, a phosphonic acid derivative, is a broad spectrum antibiotic and acts by irreversibly blocking bacterial cell wall synthesis. Fosfomycin exhibits in vitro activity against a broad range of gram-positive and gram-negative aerobic microorganisms including those associated with uncomplicated urinary tract infections. Fosfomycin is given orally as the tromethamine or calcium salt.

Fosfomycin tromethamine is approved by the FDA for the treatment of uncomplicated urinary tract infections (acute cystitis) in women due to susceptible strains of Escherichia coli and Enterococcus faecalis. Due to high dose, the tromethamine salt is not available as tablets and is provided as single-dose sachet of granules for administration as an oral solution. Another salt, fosfomycin calcium is slightly soluble in water and has lower bioavailability (Bergan T, Infection, 1990, 18, Suppl 2:S65-9). Fosfomycin disodium, used intramuscularly or intravenously, is known to cause gastric irritation (Borsa et al, Antimicrobial Agents And Chemotherapy, June 1988, Vol. 32, No. 6, p. 938-941) and is not currently available for oral administration.

There is an existing and continual need in the art for new and improved formulations that provide fosfomycin for the treatment of bacterial infections. The present invention provides novel compositions and dosage forms comprising fosfomycin disodium that are designed to minimize gastric irritation, allow flexible dose titration and increase patient compliance.

### SUMMARY OF THE INVENTION

According to some embodiments, the present invention provides compositions comprising a therapeutically effective amount of fosfomycin or a pharmaceutically acceptable salt thereof (e.g., fosfomycin disodium) for oral administration.

According to some embodiments, the present invention provides methods for treating bacterial infection by administering to a patient in need thereof, a therapeutically effective amount of fosfomycin or a pharmaceutically acceptable salt thereof (e.g., fosfomycin disodium) alone or in combination with an antibacterial agent.

### DETAILED DESCRIPTION OF THE INVENTION

The present invention provides compositions comprising a therapeutically effective amount of fosfomycin disodium for oral administration and methods for treatment of bacterial infections by administering the compositions.

In one aspect, the present invention provides oral dosage forms including immediate release, modified release, delayed release formulations and/or combinations thereof that comprise fosfomycin disodium. In some embodiments, the formulations may be immediate release. In other embodiments, the formulations may be extended release. In still other embodiments, the formulations may be delayed release.

In some embodiments, the present invention provides formulations that may include about 20% to about 95% fosfomycin (by weight). In exemplary embodiments, fosfomycin is present in an amount ranging from about 45% w/w to about 95% w/w. The formulations may comprise about 500 mg to 3 g fosfomycin. In exemplary embodiments, the formulations may comprise 1000 mg fosfomycin.

In further embodiments, the compositions may comprise a filler, a binder, an organic acid, an anti-adherent, a lubricant, a film coat or combinations thereof. The filler may be present in an amount ranging from about 0.5% to 30% and includes but is not limited to lactose, sucrose, mannitol and sorbitol. For example the compositions may comprise about 1 to 12% lactose. The binder may be present in an amount ranging from about 1% to 4% and includes but is not limited to sugars, e.g., sucrose and glucose, natural binders, e.g., starch and pre-gelatinized starch and binders such as HPMC, povidone, PEG and polyvinyl alcohol. In exemplary embodiments, the compositions comprise about 1% w/w to about 4% w/w povidone. The organic acid may be present in an amount ranging from about 0.5% w/w to about 5% w/w and includes but is not limited to citric acid, tartaric acid, succinic acid, malic acid and fumaric acid. The anti-adherent may be present in an amount ranging from about 0.5% w/w to about 2% w/w and includes but is not limited to talc and fumed silica. The lubricant may be present in an amount ranging from about 0.5% w/w to about 1% w/w and includes but is not limited to sodium stearyl fumarate. The film coat may be present in an amount ranging from about 0.5% w/w to about 2% w/w and includes but is not limited to Opadry.

Suitable plasticizers include but are not limited to polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In exemplary embodiments, the plasticizer is triethyl citrate. The concentration of the plasticizer may be about 0 to about 30%, preferably about 0 to about 10 wt % and more preferably, about 0 to about 1 wt %. In exemplary embodiments, the plasticizer may be present in an amount ranging from about 0.5% w/w to about 1% w/w.

In some embodiments, the formulations release more than about 80% fosfomycin or a pharmaceutically acceptable salt thereof (e.g., fosfomycin disodium) within 60 minutes upon entry in a use environment. For example, the formulations may release more than about 80% fosfomycin within about 10 minutes, about 15 minutes, about 30 minutes, about 45 minutes or about 60 minutes. In some embodiments, entry into a use environment includes but is not limited to contact of a formulation of the invention with the gastric or enteric fluids of a patient to whom it is administered, or with a fluid intended to simulate gastric fluid. For example, the use environment includes, but is not limited to dissolution media (e.g., pH 1.2-6.8) commonly used for testing the dissolution rate of formulations. In some embodiments, use environment refers to the stomach or other portion of the gastrointestinal tract intended as the site of major absorption locus. The fosfomycin or disodium salt may be released in a dissolution medium with a pH ranging from about 1.2 to 6.8. In exemplary embodiments, a dissolution medium of pH 6.8 is employed to simulate intestinal fluid. In other embodiments, a dissolution medium of pH 1.2 is employed to simulate gastric fluid. In some examples, the dissolution medium may be maintained at about 37° C±1° C.

In exemplary embodiments, the compositions are immediate release and provide a dissolution rate of≧80% fosfomycin after about 30 minutes at pH 1.2.

In exemplary embodiments, the compositions are delayed release compositions and comprise an enteric polymer. The enteric polymer is present in an amount ranging from about 3% w/w to about 5% w/w. Examples of enteric polymer include but are not limited to methacrylic acid copolymer, polyvinyl acetate phthalate and cellulose acetate phthalate.

In some embodiments, the compositions are modified release and comprise a pharmaceutically acceptable polymeric carrier (coating and/or matrix) that substantially contributes to the modification of the release of fosfomycin and one or more excipients. The compositions may provide improved tolerability profile upon administration to a patient in need thereof. In exemplary embodiments, the compositions provide≦10% fosfomycin release in acid and≧80% fosfomycin release in pH 6.8 buffer.

In exemplary embodiments, the present invention provides modified release solid oral dosage forms that provide dissolution rate of≦80% fosfomycin from the immediate release layer in less than 30 minutes at pH 1.2. In specific embodiments, the dissolution from the enteric coated core meets the USP criteria of≧10% release in acid and≧80% release in pH 6.8 buffer.

In exemplary embodiments, the present invention provides modified release solid oral dosage forms that provide a dissolution rate of≧80% fosfomycin from the immediate release granules in less than 30 minutes at pH 1.2. In specific embodiments, the dissolution from the enteric coated pellets meets the USP criteria of≦10% release in acid and≧80% release in pH 6.8 buffer.

In one aspect, the present invention provides pharmaceutical compositions comprising a therapeutically effective amount of fosfomycin or a pharmaceutically acceptable salt thereof (e.g., fosfomycin disodium) alone or in combination with an antibacterial agent.

In another aspect, the present invention provides methods of treating bacterial infection by administering a therapeutically effective amount of fosfomycin or a pharmaceutically acceptable salt thereof (e.g., fosfomycin disodium) alone or in combination with an antibacterial agent to a patient in need thereof. The combinations are safe and effective for the treatment of bacterial infections. In some embodiments, the combination is synergistic in treating specific infections.

Examples of bacterial infections include but are not limited to cystitis, pyelonephritis, perinephric abscess, enteritis, endocarditis, osteomyelitis, nosocomial infections (e.g., nosocomial pneumonia), cystic fibrosis, osteoarthritis, lung infections, bacteremia and diabetic foot infections (DFI).

In exemplary embodiments, the bacterial infection is an uncomplicated urinary tract infection (acute cystitis). In some embodiments, the bacterial infection is a complicated urinary tract infection. In other embodiments, the bacterial infection is an extended spectrum β-lactamase (ESBL) cystitis.

In exemplary embodiments, the bacterial infection may be due to gram negative, gram positive, aerobic or anaerobic microorganisms that are susceptible to fosfomycin or a pharmaceutically acceptable salt, solvate or prodrug thereof (e.g., fosfomycin disodium). The bacterial infection may be due to species or strains belonging to genera such as Klebsiella, Pseudomonas, Escherichia, Staphylococcus, Citrobacter, Enterococcus, Proteus, Serratia, Acinetobacter, Haemophilus and Providencia. For example, the bacterial infection may be due to Escherichia coli and Enterobacter species. In specific embodiments, the infection may be due to ESBL-producing Enterobacteriaceae (for example, Escherichia coli) or KPC enterobacteriaceae. In some embodiments, the infection may be due to carbapenem-resistant enterobacteriaceae (CRE).

In exemplary embodiments, the infection may be due to Citrobacter diversus, Citrobacter freundii, Pseudomonas aeruginosa, Enterobacter aerogenes, Enterobacter cloacae, Klebsiella oxytoca, Klebsiella pneuomoniae, Proteus mirabilis, Proteus vulgaris, Enterococcus faecium, Serratia marcescens, Acinetobacter spp. (e.g., A. baumannii), Haemophilus influenzae, Providencia rettgeri, Staphylococcus saprophyticus, methicillin-resistant Staphylococcus aureus or vancomycin-resistant enterococci. In other embodiments, the infection may be due to Enterococcus faecalis.

In some embodiments, the methods comprise administering compositions described herein. The methods include single dose as well as multi-dose therapy (e.g., 3 to 5 days). In exemplary embodiments, a dose equivalent to 3 g fosfomycin is administered. The dose may be given once, twice or thrice daily depending on the infection. In exemplary embodiments, the treatment comprises oral administration of a single dose equivalent to 3 g fosfomycin. The dose and duration of treatment may depend on the type and severity of target infection. In some embodiments, a dose of about 2-4 g may be administered to adults or a dose of about 100-250 mg/kg may be administered to infants and children. The compositions described herein provide a Cmax ranging from about 10 to 50 μg/mL after single dose administration with or without food. In some embodiments, the bioavailability may be reduced under fed conditions.

In some embodiments, methods to determine susceptibility of suspected microorganisms may be used prior to administration of fosfomycin or a pharmaceutically acceptable salt thereof. For example, quantitative methods that require measurement of zone diameters maybe employed to get reproducible estimates of the susceptibility of bacteria to antimicrobial agents including fosfomycin disodium or combinations described herein. One example is the use of standardized inoculum concentrations using paper disks comprising fosfomycin or combinations to test the susceptibility of microorganisms.

In some embodiments, the antibacterial agent, may include but is not limited to β-lactams, aminoglycosides, tetracyclines, sulfonamides, trimethoprim, fluoroquinolones, vancomycin, macrolides, polymyxins, chloramphenicol and lincosamides.

In exemplary embodiments, the antibacterial agent may be daptomycin or a pharmaceutically acceptable salt, solvate or prodrug thereof For example, the combination of fosfomycin and daptomycin may be administered to patients with osteomyelitis (e.g., due to MRSA). In other embodiments, the antibacterial agent may be a carbapenem selected from the group consisting of imipenem, biapenem, meropenem, ertapenem, faropenem, doripenem, panipenem and PZ-601. In exemplary embodiments, the antibacterial agent may be imipenem. In other embodiments, the antibacterial agent may be doripenem.

In exemplary embodiments, the antibacterial agent may be trimethoprim. In other embodiments, the antibacterial agent is trimethoprim/sulfamethoxazole. In still other embodiments, the antibacterial agent is nitrofurantoin.

In exemplary embodiments, the antibacterial agent may be a polymyxin antibiotic such as colistin.

In yet other embodiments, the methods may comprise administering fosfomycin and a fluoroquinolone, including, but not limited to, levofloxacin, ciprofloxacin, ofloxacin, gatifloxacin, norfloxacin, moxifloxacin and trovafloxacin. In particular embodiments, the fluoroquinolone may be ciprofloxacin or levofloxacin or a pharmaceutically acceptable salt, solvate or prodrug thereof.

In some embodiments, the antibacterial agent may be amoxicillin, amoxicillin, amoxicillin-clavulanate, tigecycline, temocillin, perfloxacin, pipedemic acid, ceftriaxone, mecillinam, tobramycin, piperacillin-tazobactam or combinations thereof. In exemplary embodiments, fosfomycin may be combined with ceftriaxone to treat infective endocarditis. In other embodiments, fosfomycin may be combined with tobramycin to treat cystic fibrosis. In still other embodiments, fosfomycin may be combined with meropenem to treat Lemierre syndrome and other infections due to Klebsiella pneumoniae.

The dose of antibacterial agent may depend on the type of infection to be treated. In some embodiments, the dose may range from about 0.1 to 100 mg/kg of body weight. In other embodiments, the dose may range from about 1 to 50 mg/kg of body weight. In still other embodiments, the dose may range from about 1 to 10 mg/kg of body weight.

In some embodiments, the dose of antibacterial agent (e.g., ciprofloxacin) may range from about 1 mg to 1000 mg. In other embodiments, the dose may range from about 10 mg to 800 mg. In still other embodiments, the dose may range from about 100 to 500 mg.

The ratio of fosfomycin to the antibacterial agent may range from 10:1 to 1:10 by weight. In specific embodiments, the ratio may range from about 4:1 to 1:4.

### Definitions

The term “pharmaceutically acceptable” means biologically or pharmacologically compatible for in vivo use in animals or humans, and preferably means approved by a regulatory agency of the Federal or a state government or listed in the U.S. Pharmacopeia or other generally recognized pharmacopeia for use in animals, and more particularly in humans

The term “prodrug” means a compound that is a drug precursor, which upon administration to a subject undergoes chemical conversion by metabolic or chemical processes to yield a compound, which is an active moiety.

The terms “treat,” “treatment,” and “treating” refer to one or more of the following: relieving or alleviating at least one symptom of a bacterial infection in a subject; relieving or alleviating the intensity and/or duration of a manifestation of bacterial infection experienced by a subject; and arresting, delaying the onset (i.e., the period prior to clinical manifestation of infection) and/or reducing the risk of developing or worsening a bacterial infection.

An “effective amount” means the amount of a composition according to the invention that, when administered to a patient for treating an infection or disease is sufficient to effect such treatment. The “effective amount” will vary depending on the active ingredient, the state, infection, disease or condition to be treated and its severity, and the age, weight, physical condition and responsiveness of the mammal to be treated.

The term “therapeutically effective” applied to dose or amount refers to that quantity of a compound or pharmaceutical composition that is sufficient to result in a desired activity upon administration to a mammal in need thereof.

A subject or patient in whom administration of the therapeutic compound is an effective therapeutic regimen for an infection or disease is preferably a human, but can be any animal, including a laboratory animal in the context of a trial or screening or activity experiment. Thus, as can be readily appreciated by one of ordinary skill in the art, the methods, compounds and compositions of the present invention are particularly suited to administration to any animal, particularly a mammal, and including, but by no means limited to humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%. Alternatively, particularly with respect to biological systems or processes, the term can mean within an order of magnitude, preferably within 5-fold, and more preferably within 2-fold, of a value.

### EXAMPLES

### Example 1

Immediate release formulations comprising fosfomycin uniformly distributed in a matrix can be prepared as described below.

Fosfomycin may be granulated by using methods such as slugging, low or high shear granulation or fluid bed granulation. For example, for high shear granulation (e.g., Diosna Mixer Granulator), fosfomycin can be blended with a water soluble filler (e.g., lactose, sucrose, mannitol and sorbitol) and granulated with binder (sugars like sucrose and liquid glucose; natural binders like starch and pre-gelatinized starch; synthetic/semisynthetic polymers like HPMC, povidone, PEG, polyvinyl alcohol) solution. The granules are dried, milled and blended with an organic acid (e.g., citric acid, tartaric acid, succinic acid, malic acid, fumaric acid), anti-adherent (e.g., talc, fumed silica) and lubricant (sodium stearyl fumarate). The organic acid serves as a neutralizer counteracting microenvironmental high alkalinity resulting from fosfomycin sodium. Fosfomycin sodium microenvironmental pH of about 9 would be reduced to pH 6-8 due to the presence of an organic acid. The final blend is compressed into tablets (1.4-1.7 g/tablet) followed by film coating (e.g., Opadry) in a conventional coating pan. These formulations can provide≧80% fosfomycin dissolution in less than 30 minutes at pH 1.2. Table 1 provides compositions comprising fosfomycin sodium for oral administration.

TABLE 1

| Ingredient | % w/w | grams |
|---|---|---|
| Fosfomycin sodium (1000 mg eqv) | 75-95 | 750-950 |
| Filler (e.g., lactose) | 1-12 | 10-120 |
| Binder (e.g., Povidone) | 1-3 | 10-30 |
| Organic acid (e.g., Citric acid) | 1-5 | 10-50 |
| Anti-adherent (e.g., Talc) | 0.5-2 | 5-20 |
| Lubricant (e.g., Sodium stearyl fumarate) | 0.5-1 | 5-10 |
| Film coat (e.g., Opadry) | 1-2 | 10-20 |
| Purified water* | — | 50-500 |
| Total | | 1000 |

*Removed during processing

### Example 2

An enteric coated delayed release tablet formulation of fosfomycin can be prepared as described below.

Certain salts of fosfomycin are known to cause gastric irritation (e.g., fosfomycin sodium) and delayed release of such salts would prevent any irritation. The enteric coat consisting of an enteric polymer (e.g., methacrylic acid copolymers, polyvinyl acetate phthalate and cellulose acetate phthalate) dissolves at pH and thus prevents the release of fosfomycin in the stomach. The core tablet contains fosfomycin uniformly distributed in a matrix. For manufacturing the core tablet, fosfomycin is blended with a water-soluble filler (e.g., lactose) and granulated with binder solution. The granules are dried, milled and blended with organic acid, anti-adherent and a water soluble lubricant (e.g., sodium stearyl fumarate). The final blend is compressed into tablets (1.4-1.7 g/tablet) followed by a barrier coating with film coat. Delayed release property is imparted by using an enteric polymer and plasticizer (e.g., propylene glycol, PEG 400, PEG 6000, triacetin, triethyl citrate). These formulations can meet the USP dissolution criteria of≦10% release in acid and≧80% release in pH 6.8 buffer. Table 2 provides compositions comprising fosfomycin for oral administration.

TABLE 2

| Ingredient | % w/w | Grams |
|---|---|---|
| Fosfomycin (1000 mg eqv) | 75-90 | 750-900 |
| Filler (e.g., lactose) | 2-8 | 20-80 |
| Binder (e.g., Povidone) | 1.5-3 | 15-30 |
| Organic acid (e.g., citric acid) | 1-4 | 10-40 |
| Anti-adherent (e.g., Talc) | 0.5-1 | 5-10 |
| Lubricant (e.g., Sodium stearyl fumarate) | 0.5-1 | 5-10 |
| Film coat (e.g., Opadry) | 1-2 | 10-20 |
| Enteric coat (e.g., Methacrylic acid copolymer) | 3-5 | 30-50 |
| Plasticizer (e.g., Triethyl citrate) | 0.5-1 | 5-10 |
| Purified water* | — | 50-500 |
| Total | | 1000 |

*Removed during processing

### Example 3

An enteric coated delayed release tablet formulation of fosfomycin containing an immediate release component as loading dose can be prepared as described below.

Delayed release tablet would prevent any gastric irritation associated with salts of fosfomycin (e.g., fosfomycin sodium). The enteric coat dissolves at pH≧5.5 and thus prevents the release of fosfomycin in the stomach. The core tablet contains fosfomycin uniformly distributed in a matrix. Fosfomycin is blended with water soluble filler and granulated with binder solution. The granules are dried, milled and blended with an organic acid, anti-adherent and water soluble lubricant. The final blend is compressed into tablets followed by film coating. Delayed release property is imparted by using an enteric polymer and plasticizer. The enteric coated tablets are subsequently coated with an immediate release layer whereby fosfomycin, an organic acid and film coating material are dissolved in purified water and the resulting solution is sprayed onto the enteric coated cores until the required amount of fosfomycin is deposited. Final tablet weight would range from 0.7 to 1.2 g. These formulations can provide provide≧80% fosfomycin dissolution from the immediate release layer in less than 30 minutes at pH 1.2. Dissolution from the enteric coated core can meet the USP criteria of≦10% release in acid and≧80% release in pH 6.8 buffer.

Table 3 provides compositions comprising fosfomycin for oral administration.

TABLE 3

| | | |
|---|---|---|
| Ingredient | % w/w | Grams |

Core tablet

| | | |
|---|---|---|
| Fosfomycin (450 mg eqv) | 50-80 | 500-800 |
| Filler (e.g., lactose) | 4-27 | 40-270 |
| Binder (e.g., Povidone) | 1-3 | 10-30 |
| Anti-adherent (e.g., talc) | 0.5-2 | 5-20 |
| Lubricant (e.g., sodium stearyl fumarate) | 0.5-1 | 5-10 |
| Purified water* | — | 50-500 |

DR layer

| | | |
|---|---|---|
| Enteric coat (e.g., Methacrylic acid | 3-5 | 30-50 |
| copolymer) | | |
| Plasticizer (e.g., Triethyl citrate) | 0.5-1 | 5-10 |
| Anti-adherent (e.g., talc) | 0.5-1 | 5-10 |
| Purified water* | — | 300-500 |

IR layer

| | | |
|---|---|---|
| Fosfomycin (50 mg eqv) | 5-8 | 50-80 |
| Organic acid (e.g., Citric acid) | 1-3 | 10-30 |
| Film coat (e.g., Opadry) | 1-2 | 10-20 |
| Purified water* | — | 100-400 |
| Total | | 1000 |

*Removed during processing

### Example 4

Fosfomycin tablet comprising a core of enteric coated pellets and an immediate release layer surrounding the core can be prepared as described below.

The enteric coat dissolves at pH≧5.5 preventing the release of fosfomycin in the stomach and thereby protecting from any gastric irritation associated with some salts of fosfomycin (e.g., fosfomycin sodium). Fosfomycin, povidone and Opadry are dissolved in purified water and coated onto isomalt pellets. Alternatively, sugar or micro crystalline cellulose spheres can also be used for drug layering. The drug layered pellets are then coated with an enteric layer comprising methacrylic acid copolymer and triethyl citrate. Subsequently, the enteric coated pellets are further coated with a top coat of Opadry. The final coated pellets are blended with micro crystalline cellulose (as cushioning agent), sodium stearyl fumarate and talc followed by compression. An immediate release layer is deposited on the compressed tablet by coating with an aqueous solution of fosfomycin, citric acid and Opadry. Final tablet weight would range from 1 to 1.3 g.

These formulations can provide provide≧80% fosfomycin dissolution from the immediate release layer in less than 30 minutes at pH 1.2. Dissolution from the delayed release core can meet the USP criteria of≦10% release in acid and≧80% release in pH 6.8 buffer. Table 4 provides compositions comprising fosfomycin for oral administration.

TABLE 4

| | | |
|---|---|---|
| Ingredient | % w/w | Grams |

Enteric coated pellet core

| | | |
|---|---|---|
| Fosfomycin (450 mg eqv) | 45-63 | 450-630 |
| Pellets (e.g., Isomalt) | 15-21 | 150-210 |
| Film coat (e.g., Opadry) | 1-4 | 10-40 |
| Binder (e.g., Povidone) | 1-4 | 10-40 |
| Enteric coat (e.g., Methacrylic acid copolymer) | 3-5 | 30-50 |
| Plasticizer (e.g., Triethyl citrate) | 0.5-1 | 5-10 |
| Filler (e.g., lactose) | 1-15 | 10-15 |
| Lubricant (e.g., Sodium stearyl fumarate) | 0.5-1 | 5-10 |
| Anti-adherent (e.g., Talc) | 0.5-1 | 5-10 |
| Purified water* | — | 50-500 |

IR layer

| | | |
|---|---|---|
| Fosfomycin (50 mg eqv) | 5-7 | 50-70 |
| Organic acid (e.g., Citric acid) | 0.5-2 | 5-20 |
| Film coat (e.g., Opadry) | 1-2 | 10-20 |
| Purified water* | — | 100-400 |
| Total | | 1000 |

*Removed during processing

### Example 5

Fosfomycin tablet comprising enteric coated pellets and immediate release granules can be prepared as described below.

The enteric coat allows release of fosfomycin only in the intestine and thus protects from any gastric irritation associated with some salts of fosfomycin (e.g., fosfomycin sodium). Drug layered pellets are manufactured by coating an aqueous solution of fosfomycin, binder and film coat onto pellets (e.g., isomalt). Alternatively, sugar or micro crystalline cellulose spheres can also be used for drug layering. The drug layered pellets are then coated with an enteric layer and plasticizer. Subsequently, the enteric coated pellets are further coated with a top coat. For the manufacturing of immediate release granules, fosfomycin is blended with a water soluble filler and organic acid and granulated with binder solution. Organic acid counteracts any high alkalinity as might be caused by certain salts of fosfomycin (e.g., fosfomycin sodium). The granules are dried, milled and blended with the enteric coated pellets along with water soluble filler, anti-adherent and water soluble lubricant. The final blend is compressed into tablets followed by film coating in a conventional coating pan. Alternatively, the IR granules and enteric coated pellets can be filled into capsules. These formulations can provide≧80% fosfomycin dissolution from the immediate release granules in less than 30 minutes at pH 1.2. Dissolution from the enteric coated pellets can meet the USP criteria of≦10% release in acid and≧80% release in pH 6.8 buffer. Table 5 provides compositions comprising fosfomycin for oral administration.

TABLE 5

| | | |
|---|---|---|
| Ingredient | % w/w | Grams |

Enteric coated pellets

| | | |
|---|---|---|
| Fosfomycin (450 mg eqv) | 45-63 | 450-630 |
| Pellets (e.g., Isomalt) | 15-21 | 150-210 |
| Film coat (e.g., Opadry) | 1-4 | 10-40 |
| Binder (e.g., Povidone) | 1-3 | 10-30 |
| Enteric coat (e.g., Methacrylic acid copolymer) | 3-5 | 30-50 |
| Plasticizer (e.g., Triethyl citrate) | 0.5-1 | 5-10 |
| Purified water* | — | 50-500 |

IR granules

| | | |
|---|---|---|
| Fosfomycin (50 mg eqv) | 5-7 | 50-70 |
| Filler (e.g., Lactose) | 0.5-2 | 5-20 |
| Binder (e.g., Povidone) | 0.3-0.5 | 3-5 |
| Organic acid (e.g., Citric acid) | 0.5-1 | 5-10 |
| Purified water | — | 10-200 |

Final tablets

| | | |
|---|---|---|
| Filler (e.g., Lactose) | 1-15 | 10-150 |
| Lubricant (e.g., Sodium stearyl fumarate) | 0.5-1 | 5-10 |
| Anti-adherent (e.g., Talc) | 0.2-0.5 | 2-5 |
| Film coat (e.g., Opadry) | 0.5-2 | 5-20 |
| Total | | 1000 |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

All patents, patent applications, publications, product descriptions, and protocols are cited throughout this application, the disclosures of which are incorporated herein by reference in their entireties for all purposes.

What is claimed is:

1. A pharmaceutical composition comprising a therapeutically effective amount of fosfomycin disodium for oral administration.

2. The composition according to claim 1, wherein the composition further comprises a filler.

3. The composition according to claim 2, wherein the filler is present in an amount from about 1% w/w to about 27% w/w.

4. The composition according to claim 2, wherein the filler is selected from the group consisting of lactose, sucrose, mannitol and sorbitol.

5. The composition according to claim 1, wherein the composition further comprises a binder.

6. The composition according to claim 5, wherein the binder is present in an amount from about 1% w/w to about 4% w/w.

7. The composition according to claim 5, wherein the binder is a sugar.

8. The composition according to claim 7, wherein the sugar is selected from the group consisting of sucrose and glucose.

9. The composition according to claim 5, wherein the binder is a natural binder.

10. The composition according to claim 9, wherein the natural binder is selected from the group consisting of starch and pre-gelatinized starch.

11. The composition according to claim 5, wherein the binder is selected from the group consisting of HPMC, povidone, PEG and polyvinyl alcohol.

12. The composition according to claim 5, wherein the binder is povidone.

13. The composition according to claim 12, wherein the povidone is present in an amount from about 1% w/w to about 4% w/w.

14. The composition according to claim 1, wherein the composition further comprises an organic acid.

15. The composition according to claim 14, wherein the organic acid is present in an amount from about 0.5% w/w to about 5% w/w.

16. The composition according to claim 14, wherein the organic acid is selected from the group consisting of citric acid, tartaric acid, succinic acid, malic acid and fumaric acid.

17. The composition according to claim 1, wherein the composition further comprises an anti-adherent.

18. The composition according to claim 17, wherein the anti-adherent is present in an amount from about 0.5% w/w to about 2% w/w.

19. The composition according to claim 17, wherein the anti-adherent is talc.

20. The composition according to claim 1, wherein the composition further comprises a lubricant.

21. The composition according to claim 20, wherein the lubricant is present in an amount from about 0.5% w/w to about 1% w/w.

22. The composition according to claim 20, wherein the lubricant is sodium stearyl fumarate.

23. The composition according to claim 1, wherein the composition further comprises a film coat.

24. The composition according to claim 23, wherein the film coat is present in an amount from about 0.5% w/w to about 2% w/w.

25. The composition according to claim 23, wherein the film coat is opadry.

26. The composition according to claim 1, wherein the composition further comprises an enteric coat.

27. The composition according to claim 26, wherein the enteric coat is present in an amount from about 3% w/w to about 5% w/w.

28. The composition according to claim 26, wherein the enteric coat is methacrylic acid copolymer.

29. The composition according to claim 1, wherein the composition further comprises a plasticizer.

30. The composition according to claim 29, wherein the plasticizer is present in an amount from about 0.5% w/w to about 1% w/w.

31. The composition according to claim 29, wherein the plasticizer is triethyl citrate.

32. The composition according to claim 1, wherein the composition is delayed release and comprises an enteric polymer.

33. The composition according to claim 32, wherein the enteric polymer is present in an amount from about 3% w/w to about 5% w/w.

34. The composition according to claim 32, wherein the enteric polymer is selected from the group consisting of methacrylic acid copolymer, polyvinyl acetate phthalate and cellulose acetate phthalate.

35. The composition according to claim 1, wherein fosfomycin disodium is present in an amount from about 45% w/w to about 95% w/w.

36. The composition according to claim 1, wherein the composition provides a dissolution rate of≧80% fosfomycin after about 30 minutes at pH 1.2.

37. The composition according to claim 1, wherein the composition provides a dissolution rate of≦10% fosfomycin release in acid and≧80% fosfomycin release in pH 6.8 buffer.

38. The composition according to claim 1, wherein the composition provides a dissolution rate of≧80% fosfomycin from an immediate release layer in less than 30 minutes at pH 1.2.

39. The composition according to claim 1, wherein the composition comprises immediate release granules and provides a dissolution rate of≧80% fosfomycin from the immediate release granules in less than 30 minutes at pH 1.2.

40. A method of treating a bacterial infection comprising administering the pharmaceutical composition of claim 1 to a patient in need thereof.

41. The method according to claim 40, wherein the infection is a urinary tract infection.

42. The method according to claim 40, wherein the infection is an uncomplicated urinary tract infection.

43. The method according to claim 40, wherein the infection is a urinary tract infection due to carbapenemase-resistant Enterobacteriaceae.

44. The method according to claim 40, wherein the infection is cystitis due to an extended spectrum beta lactamase producing microorganism.

45. The method according to claim 40, further comprising administering an antibacterial agent.

46. The method according to claim 45, wherein the antibacterial agent is selected from the group consisting of daptomycin, doripenem, imipenem, trimethoprim, levofloxacin and ciprofloxacin.
